(19) United States
(12) Patent Application Publication
(10) Pub. No.: US 2005/0138677 A1
(43) Pub. Date: Jun. 23, 2005

(54) Transgenic animal model for the treatment of skin tumors

(76) Inventors: Herbert J. Pfister, Koln (DE); Inke D. Schaper, Koln (DE); Pawel G. Fuchs, Koln (DE)

Correspondence Address: CLARK & ELBING LLP, 101 FEDERAL STREET, BOSTON, MA, 02110, US

(21) Appl. No.: 10/942,473

(22) Filed: Sep. 16, 2004

Related U.S. Application Data
(60) Provisional application No. 60/503,408, filed on Sep. 16, 2003.

(51) Int. Cl. A01K 67/027

(52) U.S. Cl. 800/9; 800/14; 800/18

(57) ABSTRACT

A novel animal model for the treatment of skin tumors is described. In particular, the invention provides transgenic non-human animals comprising a recombinant nucleic acid molecule containing a nucleic acid sequence encoding at least one of the gene products of the early genes of a virus of the Human Papilloma Virus Group B1, in which the animal displays one or more clinical symptoms of a tumor. The transgenic animals can be used to screen anti-tumor agents and to identify the tumorigenic potential of compounds.

### CROSS REFERENCE TO RELATED APPLICATIONS

This application claims priority U.S. Provisional Application No. 60/503,408, filed on Sep. 16, 2003, which is incorporated by reference in its entirety.

### FIELD OF THE INVENTION

The present invention relates to non-human animals that have been modified to express early genes of viruses of the Human Papilloma Virus Group B1. The animals display one or more clinical symptoms of a tumor and can serve as an animal model for tumor related disorders. Furthermore, the present invention relates to the use of the animals, as well as to cells and tissue derived therefrom, for screening anti-tumor agents or for the identification of tumor promoting effects of a compound.

### BACKGROUND OF THE INVENTION

The most common malignancies in clinical practice arise in epithelia. An epithelium is the lining of a body surface that is exposed to the outside world, which places this tissue at risk for repeated damage from a variety of agents in the environment. Environmental carcinogens are the main suspects as major contributors to the development and spread of epithelial cancers. Examples of common epithelial cancers include lung, colon, and breast cancer, however, non-melanoma skin cancer (NMSC) is the most common cancer among Caucasians. It outnumbers the total of all other cancers, and it is increasing in incidence in many areas. The two main histologic types of NMSC are squamous cell carcinoma (SCC) and basal cell carcinoma (BCC). BCCs are about four times more common than SCCs in Caucasians, whereas SCCs are more frequent in blacks. The main risk factors for NMSC are exposure to UV radiation, fair skin, and the immune status of the host. Immunosuppressed organ-transplant recipients have an up to 100-fold increased risk of SCC and a 10-fold increased risk of BCC, resulting in a reversal of the normal ratio of SCC to BCC; see, e.g., Leigh et al., J. Acquir. Immune Defic. Syndr. 21 (1999), 49-57.

Although NMSC is rarely fatal due to cure rates that approach 99%, its impact on public health is, nevertheless, considerable. The annual cost of treating NMSC in the United States exceeds $500 million (Kiviat, Semin. Cancer Biol. 9 (1999), 397-403).

NMSC is associated with multiple risk factors, pathogen infection, such as viral infection, being one of them.

Infection with human papillomavirues (HPVs) is most frequently associated with benign epithelial changes. However, malignancies may develop depending on the HPV type, its persistence, and the influence of environmental factors (Pfister, Rev. Physiol. Biochem. Pharmacol. 99 (1984), 111-181). A large group of HPVs is associated with epidermodysplasia verruciformis, a lifelong disease characterized by disseminated, flat warts that develop into squamous cell carcinoma in 30 to 50% of the patients (Orth in: Salzmann and Howley (ed.) The papoviridae. Plenum Publishing Corp. (1987) 199-244, New York). The vast majority of these cancers contain the DNA of HPV5 or HPV8, which were also detected in skin carcinomas of immunosuppressed organ transplant recipients (Meyer et al., Dermatology 201 (2000), 204-211). Infection with these HPV types therefore seems to imply a high risk for malignant conversion in the course of epidermodysplasia verruciformis (EV). No single HPV types predominate in skin cancers of non-EV patients and, so far, there is no evidence of high-risk types analogous to EV or cervical cancer, where HPV16 confers a particularly high risk for malignancy. The role of HPVs in cutanous premalignant and malignant tumors has been reviewed in Pfister and Schegget, Clin. Dermatol. 15 (1997) 335-347 and Pfister, JNCI Monogr. 31 (2003) 52-56. There is no definitive therapy for EV. Experimental therapies include intralesional administration of interferons and retinoids. To date, these have resulted in only a partial or transitory effect. In advanced HPV-related carcinomas, an experimental therapy involves treatment with a combination of 13-cis retinoic acid and interferon-alpha or cholecalciferol analogues.

Thus, the goals of both HPV research and epithelial carcinoma research are similar: to identify molecules that may coordinate progression from one stage to another, to understand the role these molecules play in the advancement of the neoplastic phenotype, and to identify therapeutics that treat early stages in the neoplastic progression to prevent progression and revert affected epithelium back towards normality. There is a need in the art for tools and methods that promote these goals.

### SUMMARY OF THE INVENTION

The present invention generally relates to an animal model for skin disorders, e.g., epidermodysplasia verruciformis (EV). In particular, the present invention relates to a transgenic non-human animal comprising a recombinant nucleic acid molecule containing a nucleic acid sequence encoding at least one of the gene products of the early genes of one of the viruses of the Human Papilloma Virus (HPV) Group B1, wherein said animal displays one or more clinical symptoms of a tumor. The B1 group of HPVs comprises the cutaneous HPVs, which may lead to epithelial neoplasias. Preferably, the HPV is HPV 8.

The present invention also relates to a method of producing a transgenic non-human animal displaying one or more clinical symptoms of a tumor. The method comprises
(a) introducing a recombinant nucleic acid molecule containing a nucleic acid sequence encoding at least one of the gene products of the early genes of one of the viruses of the Human Papilloma Virus (HPV) Group B1 into an embryo of a non-human animal;
(b) implanting the embryo into a female foster animal of the same species and allowing it to develop normally until birth;
(c) screening the offspring for the presence of the nucleic acid construct in the germline; and
(d) mating the offspring whose germline contains the nucleic acid construct.

Several methods are known in the art to introduce a recombinant nucleic acid molecule into an embryo of a non-human animal. These include, for example, microinjection into a nucleus of a fertilized ovum, retroviral transfection of embryonal cells, and transfection of embryonic stem cells.

The present invention further relates to a cell line established from the transgenic animal of invention, in which the cells are capable of expressing at least one gene product of the Human Papilloma Group B1 Virus early genes. The cells may be derived from a tumor of a transgenic animal according to the invention, most preferably from a skin tumor.

Instead of propagating the modified cells in vitro they can also be transferred to another animal. The transferred cells or corresponding tissue will usually be from a tumor of the donor animal and form tumors on the transplanted host, thus providing another animal model for the study of HPV-induced cancer. Accordingly, the present invention relates to an animal model for tumor disorders, in which a tumor cell, which has a nucleic acid sequence encoding at least one gene product of the early genes of a HPV Group B1 virus stably integrated into its genome, is taken from a transgenic animal and transplanted into a host animal.

Similarly, cells and/or tissue can be cultured before being transplanted. Thus, a further embodiment of the present invention provides an animal model for tumor disorders, in which a tumor is induced upon transplantation of one or more cells of a cell line of the invention, described below, into an animal.

The present invention further relates to the use of the modified, preferably transgenic, animals for the design and screening of drugs. Generally, there are a number of approaches. One approach involves the screening of drug candidates for the prevention or treatment of a tumor disorder comprising transplanting cells from a cell line of the invention into an animal; administering one or more drug candidates to the animal; and evaluating the effect of the drug candidate(s) on the transplanted cells.

Another approach involves the screening of drug candidates for the prevention or treatment of a tumor disorder comprising the administration of one or more drug candidates directly to the transgenic animal according to the invention; and evaluating the effect of the drug candidate(s) on the animal.

A further method for the screening of drug candidates for the treatment of a tumor disorder involves an in vitro approach that comprises contacting a stable cell line of the invention with one or more drug candidates in vitro; and evaluating the effect of the drug candidate(s) on the cell line. The screening methods can be combined with each other, as well as with other screening assays known in the art.

Furthermore, the present invention relates to an anti-tumor agent identifiable by any one of the methods of the present invention, which can belong to different classes of antineoplastic drugs such as small molecules, antibodies or conjugated antibodies.

The present invention also relates to a recombinant nucleic acid molecule comprising a nucleic acid sequence encoding at least one gene product of the early genes of a Human Papilloma Group B1 Virus under the control of a regulatory sequence directing its expression in epithelial cells.

### BRIEF DESCRIPTION OF THE FIGURE

FIG. 1A shows an example for a plasmid suitable for the generation of a non-human transgenic animal of the invention. The early genes of HPV8 comprising the genomic region of nucleotides 1 to 5111 (see SEQ ID NO: 1) are ligated into an expression vector pGEM-3Z (Promega, Madison Wis.) which additionally contains the keratin-14 promoter (SEQ ID NO: 2), the second intron of the rabbit beta-globin gene (SEQ ID NO: 3) and the keratin-14 polyadenylation signal (SEQ ID NO: 4) within one reading frame; see also Vasioukhin et al., Proc. Natl. Acad. Sci. USA 96 (1999), 8551-8556. The different elements of the insert are represented by differently shaded boxes. The HPV8 genes are under transcriptional control of the keratin-14 promoter which directs expression in basal cells of the epithelium. The vector shown here was microinjected into the male pronucleus of fertilized eggs of D2B6F1Crl (DBA/B16) mice using standard techniques. Also shown are the major restriction sites used for cloning, the Sp6 and T7 sites of the vector used for control transcriptions and for sequencing, as well as the nucleotide numbers of the insert.

FIG. 1B depicts the organization of the early genes in the genome of HPV8. The genes are represented as boxes and are drawn to scale. Nucleotide 1 to 5111 of this sequence represent the HPV8-E6-L2 insert of FIG. 1A, including parts of the non-coding region.

### DETAILED DESCRIPTION OF THE INVENTION

The present invention relates to a non-human animal comprising a recombinant nucleic acid molecule containing a nucleic acid sequence encoding at least one of the gene products of the early genes of one of the viruses of the Human Papilloma Virus Group B1, in which the animal displays one or more clinical symptoms of a tumor. Preferably, the non-human animal is a transgenic animal.

The present invention is based on experiments performed in accordance with the present invention, which demonstrate for the first time the transforming potential of the early genes of a member of the HPV Group B1, i.e., HPV8, in vivo. As described in the appended examples, a transgenic mouse model has been generated by use of the complete early region of HPV8 under the control of the epithelial cell specific Keratin 14 promoter, whose activity is mainly restricted to the basal layer of the epithelium. Surprisingly, offspring of the transgenic animals spontaneously developed papillomatous, partially ulcerative and erosive skin tumors, some of them were locally restricted and some diffusely spread all over the dorsal skin. Histology revealed epidermal hyperplasia, acanthosis, hypergranulosis, mild anisonucleosis, and increased mitotic figures in suprabasal layers. Dermis and subcutis were both broadened. Thus, the tumor phenotype displayed by those animals was much more pronounced than usually found in patients suspected of being infected with HPV, which made it difficult to determine whether different diseases or susceptibility to a disease are due to or accompanied with HPV infection. The novel animal model of the present invention makes it possible to study the tumorgenic effect of compounds in the presence of HPV and, on the other hand, to evaluate potential protecting effects of compounds, which may be useful for, e.g., dermal cosmetics and therapeutics.

The gene products of the HPV early region have been well characterized in HPV16 due to its involvement in the generation of cervical cancer. Other HPVs have a similar organization. The principle transforming genes of the cancer associated HPVs are E6 and E7 (Munger et al., Cancer Surveys 12 (1992), 197-217; Iftner et al., J. Virol. 62 (1988), 3655-3661). The E6 oncoprotein targets the proteolysis of p53 through the ubiquitination pathway (Scheffner et al. Cell 63 (1990), 1129-1136), whereas the E7 protein binds the retinoblastoma protein (Dyson et al., Science, 243 (1989), 934-937) and related proteins p107 and p130 (Dyson et al. J. Virol. 66 (1992), 6893-6902), and in so doing releases E2F, a transcription factor, which transactivates several proliferation associated genes (Chellappan et al., Proc. Natl. Acad. Sci. USA, 89 (1992), 4549-4553). The remainder of the early region encodes the E2 transactivator/repressor, the E1 protein which binds to the origin of replication, the E4 protein, which has been shown to dissociate actin intermediate filaments, and the E5 protein, which increases the activity of both the EGF or PDGF receptors (Howley, (1989) Papillomaviruses and Their Replication, p. 1625-1650. In Fields et al. (ed.) Virology, 2nd Edition. Raven Press, New York).

Progression of HPV disease is associated with changes in the state of the viral genome and in patterns of viral transcription that may contribute to the development of malignancy. In condylomas, papillomas and mild/moderate dysplasias, and cancers, the virus is episomal (Crum et al., New Engl. J. Med., 310 (1984), 880-883; Cullen et al., J. Virol. 65 (1991), 606-612), and the entire early region is expressed (Shirasawa et al., J. Virol. 62 (1988), 1022-1027). In high grade dysplasias and in cancers, the viral DNA is integrated into the host genome. Integration frequently occurs in the E1/E2 ORF, disrupting the early region downstream of the E7 coding region, and potentially leading to deregulated expression of the E6 and E7 oncoproteins, due to the absence of E2 transcriptional regulation (Baker et al., J. Virol. 61 (1987), 962-971; Schwarz et al., Nature 314 (1985), 111-114; Smotkin et al., Proc. Natl. Acad. Sci. USA. 83 (1986), 4680-4684). These changes in viral structure and expression patterns during clinical progression suggest that the functions of the viral early region are necessary to initiate cellular proliferative and dysplastic changes, whereas the E6 and E7 oncoproteins may be sufficient to maintain high grade dysplasia and malignancy in HPV16.

In HPVs of the B1 group, the transforming potential of early genes appears to be different. The B1 group of human papilloma viruses (HPVs) are primarily associated with non-melanoma skin cancer (NMSC) in patients with the inherited multifactorial disease Epidermodysplasia Verruciformis (EV). Recent work suggests that they are also commonly associated with immunosuppressed renal transplant recipients. Several isolates which appear to constitute new types have been found in skin lesions of renal transplant patients (Berkhout et al., Journal of Clinical Microbiology 33 (1995), 690-695; Shamanin et al., Cancer Research 54 (1994), 4610-4613). Association of EV-related HPV types with squamous cell carcinomas (SCC) of skin, and with SCCs of the esophagus has recently been suggested. One potential new type was isolated from an immunocompetent patient (Berkhout et al., (1995)). The B1 group of HPVs includes HPV5, HPV8, HPV9, HPV12, HPV14d, HPV15, HPV17, HPV19, HPV20, HPV21, HPV22, HPV23, HPV24, HPV25, HPV36, HPV37, HPV38, HPV47, HPV49, HPV75 (VS40), HPV76 (CR148), HPVICPX1, HPVRTRX1, HPVRTRX2, HPVRTRX3, HPVRTRX4, HPVRTRX5, HPVRTRX6, HPVVS20, HPVVS42, HPVVS73, HPVVS75, HPVVS92, HPVVS102 and HPVTogawa.

Attempts to identify mechanisms by which cutaneous HPV can contribute to NMSC development revealed a rather weak transforming potential in vitro. The E6 gene of EV-HPV seems to be the dominant oncogene in rodent cells, leading to morphologic transformation and anchorage-independent growth but not to tumorigenicity in nude mice. The E7 genes of HPV types 5 and 8 were able to transform rodent cells in collaboration with an activated H-ras gene. In contrast to the E6 proteins of HPV types 16 and 18, the E6 proteins of EV-HPV do not bind the cellular p53 protein and do not promote its proteolytic degradation. Furthermore, the E7 proteins of EV-HPV interact poorly with the retinoblastoma protein pRb (reviewed in Pfister and Ter Schegget, Clin. Dermatol. 15 (1997), 335-347). So far, it has not been possible to immortalize primary human foreskin keratinocytes with DNA of HPV type 5 or 8. Retroviral transduction of E6-7 only slightly altered keratinocyte differentiation in organotypic culture (Boxman, et al., J. Invest. Dermatol. 117 (2001), 1397-1404).

An important contribution to NMSC development may be expected from the inhibition of apoptosis by E6 proteins of cutaneous HPV. Elimination of cells with heavy damage to DNA after exposure to the UVB component of sunlight is essential, since somatic mutations due to error-prone repair or oxidative damage may eventually lead to cancer. UVB radiation exposure of the skin leads to increased levels of the proapoptotic cellular Bak protein independent of p53 function. The E6 proteins of HPV types 5, 10, and 77, in turn, have been shown to target Bak for proteolytic degradation and to inhibit effectively UVB-induced apoptosis (Jackson et al., Genes Dev. 14 (2000), 3065-3073; Jackson and Storey, Oncogene 19 (2000), 592-598).

With the animal model of the present invention, i.e., the provision of a transgenic non-human animal comprising a recombinant nucleic acid molecule containing a nucleic acid sequence encoding at least one of the gene products of the early genes of one of the viruses of the Human Papilloma Virus Group B1, wherein said animal displays one or more clinical symptoms of a tumor, it is now possible to study the above-mentioned questions, such as the mechanisms underlying the transforming potential of HPV and the tumorigenic effects of compounds or environmental stress such as radiation, UV, micro- or radiowave, etc. Likewise, corresponding protecting effects can be studied, which may lead to the discovery of compounds useful in skin cosmetics, etc. Preferably, the virus of the HPV group B1 is associated with EV.

As used herein the term “early genes” refers not only to the genes expressed early in the life cycle of HPV, E1 to E7, but also fragments of the non-coding region (NCR) and the L2 reading frame. Different genes recombinantly expressed will provide animal models for different types and grades of dysplasia and malignancy.

The term “transgenic non-human animal” comprises any non-human animal or mammal having a tissue in which cells express products of the early genes of HPVs of the B1 group. Such non-human animals include vertebrates, such as non-human primates, bovine, canine, rattus and murine species, as well as rabbit and the like. Preferred non-human animals are selected from the rodent family including rat, guinea pig, and most preferably, mouse.

In preferred embodiments, the animal is a rodent, preferably a rat or a mouse.

The term “tumor” refers to benign as well as to malignant neoplasias in their respective stages. The first stage of neoplastic progression is an increased number of relatively normal appearing cells, the hyperplastic stage. There are several stages of hyperplasia in which the cells progressively accumulate and begin to develop an abnormal appearance, which is the emergence of the dysplastic phase. In epithelial dysplasias cells resemble immature epithelial cells, and during this phase of epithelial neoplastic progression, an increasing percentage of the epithelium is composed of these immature cells. Eventually, invasive cancers develop in epithelia severely affected by dysplasia.

In a preferred embodiment of the present invention the nucleic acid sequence in the recombinant nucleic acid molecule present in the non-human animal is operably linked to a regulatory sequence directing its expression to epithelial cells. “Operably linked” when describing the relationship between two polynucleotide sequences, means that they are functionally linked to each other. For example, a promoter is operably linked to a coding sequence if it controls the transcription of the sequence. As a regulatory sequence commonly used promoter elements as well as enhancers may be used. Generally, such expression regulation sequences are derived from genes that are expressed primarily in the tissue or cell type chosen. Preferably, the genes from which these expression regulation sequences are obtained are expressed substantially only in the tissue or cell type chosen, although secondary expression in other tissue and/or cell types is acceptable if expression of the recombinant DNA in the transgene in such tissue or cell type is not detrimental to the transgenic animal.

The recombinant nucleic acid molecules will usually also comprise downstream expression regulation sequences to supplement tissue or cell-type specific expression. The downstream expression regulation sequences include polyadenylation sequences (either from the endogenous gene or from other sources) and sequences that may affect RNA stability as well as enhancer and/or other sequences which enhance expression.

In a preferred embodiment, the regulatory sequence comprises an epithelial cell specific promoter. Particularly useful for targeting the expression of HPV nucleic acid sequences to epithelial cells are the promoters from genes encoding keratin. Keratin are proteins that are expressed in epithelial tissues, and specific keratin proteins, identified by a number, e.g., keratin-5, are exclusively expressed not only in certain epithelia, but also in selected cells populating the epithelia. The epidermis is composed of layers of cells (keratinocytes) that produce specific types of keratin proteins. The basal cells produce keratin 5 and 14 (K5 and K14), whereas the more mature, terminally differentiated keratinocytes, e.g., the suprabasal keratinocytes, produce K10 and K1. Promoters from other keratin genes, such as K8 and K19, are useful in direct expression to epithelia in the bladder or intestines. The basal cell specific keratin-14 (K14) promoter has been used to overexpress the growth factor TGF-α in the epidermis (Vassar et al., Cell 64 (1991), 365-380). These animals displayed a transient, neonatal hyperproliferation that disappeared in adults. Other workers have used promoters from genes specific to suprabasal cells to express growth factors, cytokines, and oncogenes in these cells; see, Cheng et al., Genes Dev. 6 (1992), 1444-1456; Guo et al., EMBO J. 12 (1993), 973-986; Turksen, Proc. Natl. Acad. Sci. USA. 89 (1992), 5068-5072; and Vassar et al., Proc. Natl. Acad. Sci. USA 86 (1989), 1563-1567). Recently, a report appeared describing the expression of the principle oncogenes of HPV 16, e.g., the E6 and E7 open reading frames, under control of a lens α-crystalline promoter (Lambert et al., Proc. Natl. Acad. Sci. USA 90 (1993), 5583-5587), while U.S. patent U.S. Pat. No. 5,709,844 employs the keratin-14 promoter for the epithelial expression of HPV16 genes.

In a preferred embodiment, the regulatory sequence directs the expression of the nucleic acid sequence in basal epithelial cells. Therefore, a basal cell keratin promoter (e.g., K5 or K14) is preferably utilized, and the keratin-14 (K14) promoter is particularly preferred. For example, Jiang et al. (Nucl. Acids Res. 18 (1990), 247-253) identified a 300 bp controlling segment of the K14 promoter conferring epithelial-specific expression, while the K5 promoter was studied by Byrne and Fuchs (Mol. Cell. Biol. 13 (1993), 3176-3190).

Due to the above referred embodiments, i.e., the epithelial specific expression of the HPV genes, it is also preferred that the tumor displayed by the transgenic animal of the present invention is a tumor of epithelial cells, most preferably a skin tumor.

In another preferred embodiment, the transgenic animal of the invention expresses at least one gene product of the E2, E6, and/or E7 genes of any one of the Human Papilloma Group B 1 Viruses. An important contribution to NMSC development may be due to the inhibition of apoptosis by E6 proteins of cutaneous HPV. Elimination of cells with heavy damage to DNA after exposure to sunlight is essential, since somatic mutations may eventually lead to cancer. UVB radiation exposure of the skin leads to increased levels of the proapoptotic cellular Bak protein independent of p53 function. The E6 proteins of HPV types 5, 10, and 77, in turn, have been shown to target Bak for proteolytic degradation and to inhibit effectively UVB-induced apoptosis. In in vitro experiments the E7 genes of HPV types 5 and 8 were able to transform rodent cells in collaboration with an activated H-ras gene.

The early genes of HPV 8 expressed in a transgenic animal led to skin tumors, as shown in the examples. Accordingly, it is a particularly preferred embodiment of the present invention that the Human Papilloma Group B1 Virus is Human Papilloma Virus type 8 (HPV-8). A review on the regulation and replication of HPV8 and characterization of the transforming capacity of the viral E7 protein is given, for example, in the Ph.D. thesis by Akgül, B., “Regulation der Transkription und Replikation von HPV8 und Charakerisierung der transformierenden Eigenschaft des viralen E7-Proteins” (2003) University of Cologne, the disclosure content of which is incorporated herein by reference.

The present invention also relates to a method of producing a transgenic non-human animal displaying one or more clinical symptoms of a tumor. The method comprises
(a) introducing a recombinant nucleic acid molecule containing a nucleic acid sequence encoding at least one of the gene products of the early genes of one of the viruses of the Human Papillomavirus Group B1 into an embryo of a non-human animal;
(b) implanting the embryo into a female foster animal of the same species and allowing it to develop normally until birth;
(c) screening the offspring for presence of the nucleic acid construct in the germline; and
(d) mating those offspring whose germline contains the nucleic acid construct.

Several methods are known in the art to introduce a recombinant nucleic acid molecule into an embryo of a non-human animal.

Microinjection is a preferred method for transforming a zygote or early stage embryo. In the mouse, the male pronucleus reaches the size of approximately 20 micrometers in diameter which allows reproducible injection of 1-2pl of DNA solution. The use of zygotes as a target for gene transfer has a major advantage in that in most cases the injected DNA will be incorporated into the host gene before the first cleavage (Brinster et al., Proc. Natl. Acad. Sci. USA 82 (1985), 4438-4442). As a consequence, all cells of the transgenic non-human animal will carry the incorporated transgene. This will, in general, also be reflected in the efficient transmission of the transgene to offspring of the founder since 50% of the germ cells will harbor the transgene. Once the DNA molecule has been injected into the fertilized egg cell, the cell is implanted into the oviduct of a recipient female, and allowed to develop into an animal, as described in Example 1.

Retrovital infection can also be used to introduce a transgene into a non-human animal. The developing non-human embryo can be cultured in vitro to the blastocyst stage. During this time, the blastomeres can be targets for retrovital infection (Jaenisch, Proc. Natl. Acad. Sci USA 73 (1976), 1260-1264). Efficient infection of the blastomeres is obtained by enzymatic treatment to remove the zona pellucida (Hogan, et al. (1986) In Manipulating the Mouse Embryo, Cold Spring Harbor Laboratory Press, Cold Spring Harbor, N.Y.). The viral vector system used to introduce the transgene is typically a replication-defective retrovirus carrying the transgene (Jahner et al., Proc. Natl. Acad. Sci. USA 82 (1985), 6927-6931; Van der Putten et al., Proc. Natl. Acad. Sci. USA 82 (1985), 6148-6152). Transfection is easily and efficiently obtained by culturing the blastomeres on a monolayer of virus-producing cells (Van der Putten, supra; Stewart et al., EMBO J. 6 (1987), 383-388).

More recently embryonic stem (ES) cells have been employed to generate trangenic animals. ES cells are obtained from pre-implantation embryos cultured in vitro (Evans et al., Nature 292 (1981), 154-156; Bradley et al., Nature 309 (1984), 255-258; Gossler et al., Proc. Natl. Acad. Sci USA 83 (1986), 9065-9069; and Robertson et al., Nature 322 (1986), 445-448). Transgenes can be efficiently introduced into ES cells using a number of means well known to those of skill in the art. Such transformed ES cells can thereafter be combined with blastocysts from a non-human animal. The ES cells thereafter colonize the embryo and contribute to the germ line of the resulting chimeric animal; for a review see Jaenisch, Science 240 (1988), 1468-1474).

By breeding and inbreeding such animals, it is possible to produce heterozygous and homozygous transgenic animals. Animals according to the present invention include without limitation rodents, such as rats, mice, guinea pigs and gerbils, dogs, cats, pigs, sheep, cows, goats, horses, and rabbits. Transgenic animals are those which have incorporated a foreign gene into their genome. A transgene is a foreign gene or recombinant nucleic acid construct which has been incorporated into a transgenic animal.

The success rate for producing transgenic animals is greatest in mice. A number of other transgenic animals have also been produced. These include rabbits, sheep, cattle, and pigs (Jaenisch (1988), Hammer et al., J. Animal Sci. 63 (1986), 269; Hammer et al., Nature 315 (1985), 680; Wagner et al., Theriogenology 21 (1984), 29). Effective generation of transgenic pigs and mice are also described in, for example, Chang et al., BMC Biotechnol. 2 (1): 5 (2002). Generation of transgenic rabbits is described in James et al., J. Mol. Cell Cardiol. 34 (2002), 873-882 and Murakami et al., Theriogenology 57 (2002), 2237-2245. Furthermore, the generation of transgenic sheep is described for example in Kadokawa et al., Domest. Anim. Endocrinol. 24 (2003), 219-229 and Campbell, Methods Mol. Biol. 180 (2002), 289-301. U.S. Pat. No. 5,639,457 is also incorporated herein by reference to supplement the present teaching regarding transgenic pig and rabbit production. U.S. Pat. Nos. 5,175,384; 5,175,385; 5,530,179, 5,625,125, 5,612,486 and 5,565,186 are also each incorporated herein by reference to similarly supplement the present teaching regarding transgenic mouse and rat production.

Screening the offspring of an animal for the expression of a desired transgene can be done by several methods known in the art. For example RT-PCR can be employed to amplify the transgene or fragments thereof from RNA obtained from the animal, usually from tail clippings or blood. Standard PCR methods useful in the present invention are described in PCR Protocols: A Guide to Methods and Applications (Innis et at., eds., Academic Press, San Diego 1990). It is also possible to introduce a marker together with the transgene. Other techniques include protein based assays, such as ELISA, FISH, or Western blot techniques, which usually require an antibody directed against an epitope of the transgene or the marker. FISH techniques are described in e.g., Gall et al., Meth. Enzymol., 21(1981), 470-480 and Angerer et al. in Genetic Engineering: Principles and Methods Setlow and Hollaender, Eds. Vol 7, pgs 43-65, plenum Press, New York 1985).

The present invention further relates to a cell line established from the transgenic animal of invention and that is capable of expressing at least one gene product of the Human Papilloma Group B1 Virus early genes. Such a cell line can be easily obtained by methods well known in the art. Short Protocols in Cell Biology (2003, edited by Bonifacino, Dasso, Harford, Lippincott-Schwartz and Yamada, John Wiley & Sons, Inc.) provides a collection of protocols for establishing and maintaining cell lines. In a preferred embodiment the cells are derived from a tumor of a transgenic animal according to the invention, most preferably from a skin tumor.

Instead of propagating the transgenic cells in vitro they can also be transferred to another animal. The transferred cells or tissues will usually be from a tumor of the donor animal and form tumors on the transplanted host, thus providing another animal model for the study of HPV induced cancer. The present invention accordingly relates to an animal model for tumor disorders, in which a tumor from a transgenic animal which has stably integrated into the genome of its cells a nucleic acid sequence encoding at least one gene product of the early genes of a Human Papilloma Group B1 Virus is taken from the transgenic animal and transplanted into a host animal. General methods for the transfer of cells and/or tissues from donor onto host animals as well as screening assays related thereupon are known in the art, see, e.g., DE 196 37 645 and WO00/40082 which describe graft animal models for HPV and their use for evaluating and testing candidate therapeutic agents against HPV. The disclosure content of these references is incorporated herein in their entirety and can be adapted to the embodiments of the present invention. However, tissue size, cell number and transfer procedures may have to be optimized for each tumor, donor and host animal.

Similarly, cells and/or tissues can be cultured before being transplanted. Thus, a further embodiment of the present invention provides an animal model for tumor disorders, in which the tumor has been cultured and/or induced prior to transplanting one or more cells of a cell line of the invention into an animal.

One of skilled in the art would recognize that there are a number of approaches to the use of these transgenic animals for the design and screening of antineoplastic drugs. One approach involves the screening of drug candidates for the prevention or treatment of a tumor disorder comprising transplanting cells from a cell line of the invention into an animal; administering one or more drug candidates to the animal; and evaluating the effect of the drug candidate(s) on the transplanted cells.

By “drug candidate,” “candidate compound,” “test compound,” “agent,” or “therapeutic agent,” as used herein, means any molecule, e.g. a protein or pharmaceutical, i.e., a drug, with the capability of substantially inhibiting the growth of a tumor cell, e.g., a cell that has been transformed by the presence of a nucleic acid molecule having a sequence that encodes at least one of the gene products of the early genes of one of the viruses of the Human Papilloma Virus Group B1, which has been contacted with said drug candidate, candidate compound, test compound, agent, or therapeutic agent, relative to a tumor cell that has not been contacted with the drug candidate, candidate compound, test compound, agent, or therapeutic agent.

In a preferred embodiment the tumor is an epithelial tumor, most preferably a skin tumor. In the latter case initial evaluation of the effects of the tested drug will be, e.g., the visual assessment of the size and severity of the tumor. This has the additional advantage that the visual inspection of the tumor allows an immediate and continuous assessment of drug efficacy. In the case of non visible tumors drug effect evaluation will usually require the animal to be sacrificed to inspect the tumor. Neoplasias can be detected according to standard techniques well known to those of skill in the art. Such methods include apart from visual inspection (for lesions on the skin), histochemical and immunohistochemical techniques, and the like. Typically the drug candidate(s) are evaluated for their ability to inhibit the formation and/or the growth of tumors developed from the transplanted cell line.

Another approach involves the screening of drug candidates for the prevention or treatment of a tumor disorder comprising the administration of one or more drug candidates directly to the transgenic animal according to the invention; and evaluating the effect of the drug candidate(s) on the animal.

In a preferred embodiment the tumor is an epithelial tumor, most preferably a skin tumor, with the advantages for evaluating the drug effect as mentioned above. Typically the drug candidate(s) are evaluated for their ability to inhibit the formation and/or the growth of tumors developed by the transgenic animal.

Those skilled in the art will recognize that numerous modes of administration are possible. Modes of administration include, but are not limited to, topical application, intra- and subdermal injection, aerosol administration, and transdermal administration (e.g., in a carrier, such as DMSO). Of course, the selection of a particular mode of administration will reflect the particularities of the composition. Similarly where a potential therapeutic is expected to be administered topically as opposed to systemically, the potential therapeutic will be screened using a topical application.

A further method for the screening of drug candidates for the treatment of a tumor disorder involves an in vitro approach, which comprises contacting a stable cell line of the invention with one or more drug candidates in vitro; and evaluating the effect of the drug candidate(s) on said cell line.

In a preferred embodiment, the drug candidate(s) are evaluated for their ability to inhibit the growth of tumors after the transgenic cell line is transplanted into an animal.

In another embodiment the drug candidate(s) are evaluated for their ability to inhibit the growth of the cell line directly.

The screening methods of the invention are preferably performed with the drug candidates provided as a collection of compounds. In a preferred embodiment the number and/or diversity of compounds within said collection is successively reduced in repeated screening rounds. Such collections are also commercially available, for example, from Pharmacopeia, Inc. or Chemical Diversity Labs, Inc.

The present invention also envisages the combination of the screening methods. While cell based in vitro methods are more amenable to high throughput assay formats, the in vivo screening in animals will provide more precise results. Thus, a collection of drugs with high diversity can be tested in cell based assays employing high throughput techniques and after reducing the diversity in several round of high throughput screening the obtained low diversity collection is then tested in animals while reducing the diversity further until a single compound is identified.

The above-described methods can, of course, be combined with one or more steps of any of the above-described screening methods or other screening methods well known in the art. Methods for clinical compound discovery comprises for example ultrahigh-throughput screening (Sundberg, Curr. Opin. Biotechnol. 11 (2000), 47-53) for lead identification, and structure-based drug design (Verlinde and Hol, Structure 2 (1994), 577-587) and combinatorial chemistry (Salemme et al., Structure 15 (1997), 319-324) for lead optimization.

In a preferred embodiment of the screening methods, the drug candidate(s) are small molecule(s). Methods for the synthesis, optimization, and testing of small molecules are well known in the art. In a further embodiment, the drug candidate(s) are antibodies, preferably antibody conjugate(s). Antibodies play an increasing role in the treatment of disorders due to their high specificity. Once a target molecule with a critical role in the development of the particular disorder is identified, it is possible to generate either polyclonal or monoclonal antibodies capable of binding to it. The antibody can inhibit the detrimental function of the molecule either directly, for instance, by binding to the functional core, or by assigning the molecule to be degraded.

Antibodies can also be employed to transport a moiety, that is conjugated to the target molecule. Such a moiety can be, for example, a radionucleotide, that provides direct radiation to cells expressing the target molecule. If the target molecule is a specific tumor marker, only the tumor cells will receive lethal doses of radiation. In a similar manner, the conjugated moiety can be a toxin or a prodrug that is turned into an active drug by cellular metabolism, or due to the action of an additional drug administered to the subject.

Similarly, if the target molecule has a beneficial function, the antibody and/or the moiety conjugated to it can activate the beneficial function, protect it from degradation, or induce its expression.

The present invention further relates to a method of screening agents for a cancer promoting or protecting effect comprising:
(a) contacting cells from a cell line derived from an animal of the present invention, which has been modified to express at least one gene product of a member of the HPV Group B1 with the agent; or contacting an animal of the present invention as defined above with the agent; and
(c) evaluating the effect of the agent on the cells or the animal.

Agents that can be screened for their potential to promote or inhibit cancer development include, but are not limited to, therapeutic agents (or potential therapeutic agents), agents of known toxicities, such as toxins of epithelial cells, myotoxins, carcinogens, teratogens, or toxins to one or more reproductive organs. The agents can further be cosmetics, including so-called “cosmeceuticals,” industrial wastes or by-products, or environmental contaminants. They can also be animal therapeutics or potential animal therapeutics.

The animal model of the present invention can also be used to screen compounds with which human or animals come into contact, such as household products. In particular, such products which come into contact with the skin can be tested for the influence on the development of tumors, in particular, skin tumors due to the presence of HPV. Likewise, the potential protecting effect of such compounds may be detected. Household products that can be tested with the methods of the present invention include bleaches, toilet-cleaning products, blocks (such as sun blocks), washing-up liquids, soap powders and liquids, fabric conditioners, window, oven, floor, bathroom, kitchen and carpet cleaners, dishwater detergents and rinse aids, water-softening agents, descalers, stain removers, polishes, paints, paint removers, glues, solvents, varnishes, air fresheners, and moth balls and insecticides. Furthermore, chemical compositions of any part of a device, such as an electrode, and/or adhesives, paste, gel or cream, including the concentrations of the different ingredients and impurities that may be present, can be tested with the method of the present invention.

Compounds of interest encompass numerous chemical classes, though typically they are organic molecules. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Contacting the cells of a cell line with an agent will usually involve adding the agent in various concentration into the cell medium. But also insoluble materials may be tested, for instance, by coating a culture dish with the agent and seeding the cells onto or growing the cells in the presence of a material that sets the agent free.

The cells may also be grown in the presence of another type of cells or organisms, e.g., pathogens, transfected cells, parasites, and the like, that express the agent and deliver it into the growth medium.

Contacting an animal of the invention with the agent can be done by any appropriate means. It may, for instance, be applied onto the skin, injected, or given orally.

The effect of the agent on the cells or animals of the invention can be evaluated by observing visual changes, such as, e.g., the morphology of cells or the development of lesions and/or tumors on the skin of the animal. Other methods for the evaluation are well known in the art and include the observation of expression patterns, particularly of the expression of genes known to be involved in the development of cancer, by, e.g., promoting or inhibiting transformation. In the models of the invention particularly the expression of HPV genes and/or genes that interact with them is of interest. Methods for the observation of expression patterns include for example PCR techniques, Northern or Western blots, immunohistochemistry, in situ hybidization, etc. The methods are well known and a person skilled in the art will be able to adapt them to detect expression of genes of interest.

The agent to be tested can also be a combination of different agents, e.g., two or more agents suspected to promote cancer, thereby facilitating the identification of synergistic effects between the two or more agents. Another possible combination is that of a known cancer promoting agent with agent(s) suspected to have a protecting effect in order to be able to identify such a protective effect sooner.

Drugs and agents inhibiting the growth of the transformed cells may further be modified through conventional chemical, physical, and biochemical means, and may be used to produce combinatorial libraries. Known pharmacological agents may be subjected to directed or random chemical modifications, such as acylation, alkylation, esterification, amidification, etc. to produce structural analogs. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, peptidomimetics and/or computer aided design of appropriate derivatives and analogues can be used, for example, according to the methods described above. Methods for the lead generation in drug discovery also include using proteins and detection methods such as mass spectrometry (Cheng et al. J. Am. Chem. Soc. 117 (1995), 8859-8860) and some nuclear magnetic resonance (NMR) methods (Fejzo et al., Chem. Biol. 6 (1999), 755-769; Lin et al., J. Org. Chem. 62 (1997), 8930-8931). They may also include or rely on quantitative structure-action relationship (QSAR) analyses (Kubinyi, J. Med. Chem. 41 (1993), 2553-2564, Kubinyi, Pharm. Unserer Zeit 23 (1994), 281-290) combinatorial biochemistry, classical chemistry and others; see, for example, Holzgrabe and Bechtold, Pharm. Acta Helv. 74 (2000), 149-155.

The agents may also be combined with a suitable carrier. Examples of carriers and methods of formulation may be found in Remington's Pharmaceutical Sciences.

The present invention also relates to the anti-tumor agents identifiable and obtained by any one of the methods of the present invention.

The person skilled in the art will recognize that the antineoplastic compounds that may be identified by the methods provide by the present invention are often compounds that are capable of inducing apoptosis of particular cells. In a preferred embodiment, the methods of the invention thus further comprise the step of identifying a candidate that has the ability to induce death or apoptosis of cancer cells.

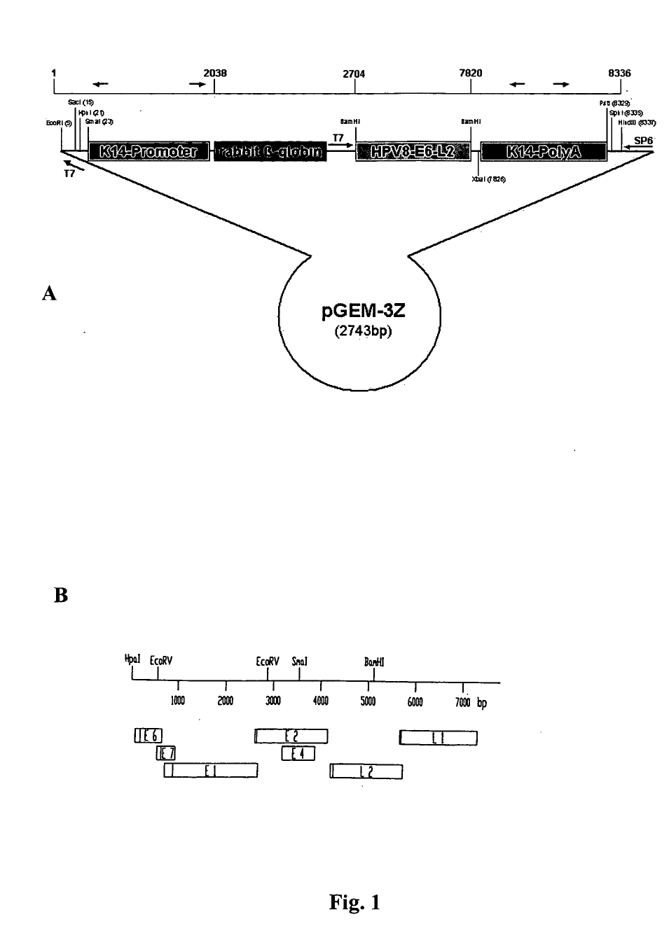

The present invention also relates to a recombinant nucleic acid molecule as defined above, i.e., comprising a nucleic acid sequence encoding at least one gene product of the early genes of a Human Papilloma Group B1 Virus under the control of a regulatory sequence directing its expression in epithelial cells. Such a recombinant nucleic acid molecule does not need to be in the form of a plasmid, as shown in Example 1 (FIG. 1); it may also be a retroviral vector or a strand of DNA with terminal repeats allowing the integration of the DNA into the host chromosome.

In a preferred embodiment of the recombinant nucleic acid molecule, the regulatory sequence comprises a keratin-14 promoter, since it directs the expression of the recombinant nucleic acid molecule in basal epithelium. It is particularly preferred, that the promoter is of human origin.

As mentioned before, novel oligonucleotides have been designed for the analysis of HPV8 transgenes. The position of each of those oligonucleotide sequences (SEQ ID NOS: 7 to 12) in the HPV8 genome (Fuchs et al., J. Virol. 58 (1986), 626-634) is as follows:
5′-Primer HPV8E6 fw: Pos. 271-292;
3′-Primer HPV8E6 bw: Pos. 404-428;
5′-Primer HPV8E7 fw: Pos. 710-734;
3′-Primer HPV8E7 bw: Pos. 848-870;
5′-Primer HPV8E2 fw: Pos. 3410-3427; and
3′-Primer HPV8E2 bw: Pos: 3567-3586. Thus, the use of an appropriate primer pair in an amplification reaction leads to the identification of DNA fragments of predefined size in case of HPV8 positive samples. In experiments performed in accordance with the present invention those primers could be shown to be particularly suitable for the detection of the presence of HPV8 DNA and mRNA, respectively, and thus make them a valuable means for diagnosis of HPV8 in subject.

Accordingly, the present invention relates to the use of an oligonucleotide for the detection of the presence of HVP8 DNA or mRNA in a sample of a subject, wherein said oligonucleotide consists of or comprises a nucleotide sequence of any one of SEQ ID NOS: 7 to 12 or a complementary strand thereof.

“Oligonucleotides” preferably refers to either a single stranded polydeoxynucleotide or two complementary polydeoxynucleotide strands that may be chemically synthesized. Such synthetic oligonucleotides have no 5′ phosphate and, thus, will not ligate to another oligonucleotide without adding a phosphate with an ATP in the presence of a kinase. A synthetic oligonucleotide will ligate to a fragment that has not been dephosphorylated.

In a particular preferred embodiment the oligonucleotide is about 10 to 100, preferably about 15 to 50, and most preferably 18 to 25 nucleotides in length, and comprises the nucleotide sequence of any one of SEQ ID NOS: 7 to 12 or a complementary sequence of any one of those. However, one or more nucleotide substitutions in the nucleotide sequences of the SEQ ID NOS may be tolerated as long as they hybridize, preferably under stringent conditions, with human papillomavirus 8 genomic sequence as described, for example, in Fuchs et al., J. Virol. 58 (1986), 626-634.

Hence, in a still further embodiment, the present invention relates to a primer or probe consisting of an oligonucleotide as defined above. In this context, the term “consisting of” means that the nucleotide sequence described above and employed for the primer or probe of the invention does not have any further nucleotide sequences of the HPV8 genomic sequence immediately adjacent at its 5′ and/or 3′ end. However, other moieties such as labels, e.g., biotin molecules, histidin flags, antibody fragments, colloidal gold, etc., as well as nucleotide sequences that do not correspond to the HPV8 genomic sequence, may be present in the primer and probes of the present invention. Furthermore, it is also possible to use the above described particular nucleotide sequences and to combine them with other nucleotide sequences derived from the HPV8 genomic sequence, wherein these additional nucleotide sequences are interspersed with moieties other than nucleic acids or wherein the nucleic acid does not correspond to nucleotide sequences of the HPV8 genomic sequence.

The oligonucleotides, probes, and primers of the present invention may be used for a variety of applications, such as PCR analysis, and may be present in a kit. The kit may comprise further components and may be packaged in containers, such as vials, optionally in buffers and/or solutions. If appropriate, one or more of the components may be packaged in the same container.

These and other embodiments are disclosed and encompassed by the description and examples of the present invention. Further literature concerning any one of the materials, methods, uses, and compounds to be employed in accordance with the present invention may be retrieved from public libraries and databases, using, for example, electronic devices. For example, the public database “Medline,” which is hosted by the National Center for Biotechnology Information and/or the National Library of Medicine at the National Institutes of Health, may be utilized. Further databases and web addresses, such as those of the European Bioinformatics Institute (EBI), which is part of the European Molecular Biology Laboratory (EMBL), are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. A more complete understanding can be obtained by reference to the following specific examples and the figures, which are provided herein for purposes of illustration only and are not intended to limit the scope of the invention.

The contents of all cited references (including literature references, issued patents, published patent applications, as cited throughout this application, and manufacturer's specifications, instructions, etc) are hereby expressly incorporated by reference; however, there is no admission that any document cited is indeed prior art as to the present invention.

The practice of the present invention will employ, unless otherwise indicated, conventional techniques of cell biology, cell culture, molecular biology, transgenic biology, microbiology, recombinant DNA, and immunology, which are all within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (D. N. Glover ed., 1985); Oligonucleotide Synthesis (M. J. Gait ed., 1984); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Gene Transfer Vectors for Mammalian Cells (Miller & Calos, eds.); Current Protocols in Molecular Biology and Short Protocols in Molecular Biology, 3rd Edition (F. M. Ausubel et al., eds.); and Recombinant DNA Methodology (R. Wu ed., Academic Press). Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.); Immobilized Cells And Enzymes (IRL Press, 1986); B. Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251);

### EXAMPLES

### Example 1

### Generation of Mice Transgenic for the Early Genes of HPV8

The early genes of HPV8 comprising the genomic region of nucleotides 1 to 5111 (Fuchs et al., J. Virol. 58, (1986) 626-634) (see SEQ ID NO: 1) were ligated into an expression vector pGEM-3Z (Promega, Madison Wis.) which additionally contained the keratin-14 promoter (SEQ ID NO: 2), the second intron of the rabbit beta-globin gene (SEQ ID NO: 3) and the keratin-14 polyadenylation signal (SEQ ID NO: 4) within one reading frame; see also Vasioukhin et al., Proc. Natl. Acad. Sci. USA 96 (1999), 8551-8556. An intron and a poyadenylation signal are advantageous for the efficient expression of the transgenic element. The HPV8 genes are under transcriptional control of the keratin-14 promoter which directs expression in basal cells of the epithelium (Vasioukhin et al., 96 (1999), 8551-8556). The vector, as shown in FIG. 1, was microinjected into into the male pronucleus of fertilized eggs of D2B6F1Crl (DBA/B16) mice using standard techniques; see, e.g. Hammes, A and Schedl, A (2000, in Mouse genetics and Transgenics—a practical approach (Jackson, I J and Abbott, C M, Eds.), Oxford University Press, New York: 217-245).

### Example 2

### Expression of HPV8 Early Genes in Transgenic Mice

To test which of the progeny mice were expressing the HPV early genes, a PCR of the early genes E6 to E7 was performed. The following primer and amplification conditions were used:

| | | |
|---|---|---|
| E6/E7 forward | 5′-CAA TTT TCC TAA GCA AAT GGA C-3′ | (SEQ ID NO: 5) |
| E6/E7 reverse | 5′-CAC TAC ATT CAG CTT CCA AAA TAC A-3′ | (SEQ ID NO: 6) |
| DNA-template | 10 μl DNA eluate from tail clippings | |
| | prepared with the Qiamp Tissue kit (Qiagen. Hilden) | |
| | according to the manufacturers intstructions | |
| HPV8-E6/7 forward | 0.3 μM | |
| HPV8-E6/7 reverse | 0.3 μM | |
| DNTPs | 0.3 mM | |
| 10x DNA-Polymerase buffer | 5 μl | |
| Taq-Polymerase (Pharmacia) | 2.5 U | |
| H₂O | ad 50 μl | |

Cycling conditions:
3 min denaturing at 95° C.;
35 cycles of 45 sec at 95° C.; 1 min annealing at 50° C.; 1.5 min elongation at 72° C.
final extension for 10 min at 72° C.
PCR products were visualized after agarose gelelectrophoresis.

Mice developing from the microinjected eggs in foster mothers were born three weeks after implantation (F0 generation) and backcrossed with FVB/N or B 16 wild type mice to generate the subsequent F 1 to F5 generations. In particular, four of the positively tested transgenic animals (No. 9, 61, 85 and 88) were used for the breeding program and mated with wild-type mice lines FVB/N and BI6, respectively. Number 9 and 85 were chosen since they displayed a tumor already at the beginning of the breeding program, while choice for number 61 and 88 was by chance. The aim of the backcrossing was to achieve a clean FVB/N and BI6 background, respectively, of the transgenic mice, which can usually be established after 6 to 8 generations. The two different types of wild-type mice were used in order to investigate the possible role of the different genetic background with respect to the susceptibility for tumor development. Therefore, line 9 and 88 were solely crossed with FVB/N mice, line 61 with BI6 mice and line 85 with both. From each new generation some of the HPV8-positive mice were used for further breeding with the appropriate wild-type mice. Until the present analysis five generations (F1-F5) could be established for line 9 and three generations (F1-F3) for lines 85, 85 BI6 and 61. All of the progeny of line 88 were HPV8-negative. The results for the HPV8-positive mice are shown in Table I below:

TABLE 1
Proportion transgene-positive mice in generations
F1-F5 of lines 9, 85, 85 BI6 and 61.
Transgene-positive mice/tested mice

| Gen- | | Line | Line 85 | |
|---|---|---|---|---|
| eration | Line 9/FVB/N) | 85/FVB/N) | (BI6) | Line 61 (BI6) |
| F1 | 25/40 (62.5) | 7/24 (29.2) | 4/12 (33.3) | 5/6 (83.3) |
| F2 | 19/38 (50) | 12/32 (37.5) | 5/7 (71.4) | 4/14 (28.6) |
| F3 | 49/82 (59.8) | 8/14 (57.1) | 2/6 (33.3) | 4/8 (50) |
| F4 | 27/49 (55.1) | | | |
| F5 | 8/17 (47.1) | | | |
| Positive | 128 (56.5%) | 27 (38.6%) | 11 (44%) | 13 (46.4%) |
| total | | | | |

In order to quantify gene expression in the transgenic mice real time PCR was performed for the E2, E6 and E7 gene using the Light Cycler System from Roche Molecular Biochemicals, Mannheim.

PCR primer for real time PCR:

SEQ ID NO: 7

| | |
|---|---|
| HPV8E6 forward | 5′-GCG GCT TTA GGT ATT CCA TTG C-3′ |

SEQ ID NO: 8

| | |
|---|---|
| HPV8E6 reverse | 5′-GCT ACA CAA CAA CAA CGA CAA CAC |
| | G-3′ |

SEQ ID NO: 9

| | |
|---|---|
| HPV8E7 forward | 5′-CCT GAA GTG TTA CCA GTT GAC CTG |
| | C-3′ |

SEQ ID NO: 10

| | |
|---|---|
| HPV8E7 reverse | 5′-CAG TTG CGT TGA CAA AAA GAC G-3′ |

SEQ ID NO: 11

| | |
|---|---|
| HPV8E2 forward | 5′-AAC AGC CAC AAC AAA CCG-3′ |

SEQ ID NO: 12

| | |
|---|---|
| HPV8E2 reverse | 5′-CGT ATC CAG GTC CAG GTC CT-3′ |

RNA was isolated from normal skin, tumor and liver of three mice, subjected to reverse transcription using standard techniques, e.g. the omniscript reverse transcription kit with oligo-dT₂₃ primer (Qiagen) and real time PCR was performed. The number of copies of the HPV8 genes was determined by normalization to copy number of the beta-actin gene according to manufacturers instructions. The following results were obtained as shown in Table 2 below:

TABLE 2
cDNA-copies or HPV8 genes per β-Actin cDNA copies

| | mouse ID | | | | | | | | |
|---|---|---|---|---|---|---|---|---|---|
| | 9/3/6/5/6* | | | 9/3/6/4** | | | 85* | | |
| | Transgene/cell | | | | | | | | |
| | 26.7 | | | 15.6 | | | 14.5 | | |
| | Material | | | | | | | | |
| Gene | skin | tumor | liver | skin | tumor | liver | skin | tumor | liver |
| E2 | 0.02 | 0.41 | 10⁻³ | 1.1085 | 0.7205 | 0.02 | 0.1349 | 1.4218 | 0 |
| E6 | 0.0006 | 0.0059 | 0 | 0.0076 | 0.0043 | 0 | 0.0009 | 0.0076 | 0 |
| E7 | 0.0011 | 0.0328 | 10⁻⁴ | 0.039 | 0.026 | 10⁻⁴ | 0.0027 | 0.0533 | 0 |
| E7/E6 | 1.92 | 5.51 | | 5.15 | 6.14 | | 3.02 | 7.05 | |
| E2/E6 | 40.95 | 69.46 | | 146 | 168.86 | | 152.6 | 188.1 | |
| E2/E7 | 21.28 | 12.6 | | 28.34 | 27.51 | | 50.5 | 26.7 | |

*= benign tumor;
**= malignant tumor

In mouse 9/3/6/4, the malignant tumor expression levels were comparable in the carcinoma and the skin without pathological findings, however, the levels were 10-50-fold higher than in normal skin of mice with benign tumors.

### Example 3

### Tumor Development in HPV8 Transgenic Mice

There were obvious differences in the occurrence of tumors in the different lines in different generations. From line 9 backcrossed with FVB/N 13 mice of the F1 generation developed tumors (52%). The average age at the beginning of tumor development was 38.6 weeks. In the F2 generation 11 mice developed tumors (57.9%). The average age at the beginning of tumor development was 17.4 weeks. In the F3 generation 14 mice developed tumors (81.6%). The average age at the beginning of tumor development was 15.5 weeks. In the F4 generation 19 mice developed tumors (70.4%). The average age at the beginning of tumor development was 12.3 weeks. In the F5 generation 6 mice developed tumors (75%). The average age at the beginning of tumor development was 7.6 weeks.

With line 9 the increase of the incidence of tumor development with proceeding generations could be demonstrated. Furthermore, the average age at the beginning of tumor development decreased with respect to the later generations.

In line 85 backcrossed with FVB/N three mice in the F2 generation developed tumors (52%). The average age at the beginning of tumor development was 22.9 weeks.

From line 85 BI6 backcrossed with BI6 one mouse developed tumors out of the F1 generation (25%). The age at the beginning of tumor development was 45.6 weeks. In the F2 generation two mice developed tumors (40%). The average age at the beginning of tumor development was 26.7 weeks. In the F3 generation two mice developed tumors (100%). The average age at the beginning of tumor development was 8.3 weeks.

In line 61 crossed with BI6 none of the mice of the F1 as well of the F3 generation developed tumors. In the F2 generation one mouse at the age of 9.3 weeks developed a tumor.

Furthermore, 31 out of 104 mice with tumors were female (29.8%) and 73 of the mice with tumors were male (70.2%). This distribution could be observed in all of the lines investigated. None of the HPV8-negative mice developed a tumor.

The following Table 3 summarizes the tumor development:

TABLE 3
Tumor development in the different generations of HPV8 transgenic mice

| | | | | |
|---|---|---|---|---|
| | | mean age | mean age | number; mean age |
| | tumor+/HPV+ | (range; std dev.) | (range; std dev.) | (range; std dev.) |
| generation | (%) | HPV+, tumor+ | HPV+, tumor− | HPV− |

line 9

| | | | | |
|---|---|---|---|---|
| F1 | 13/25 (52) | 38.6 (13.5-65.9; 16.5) | 46.5 (8.8-72.6; 28.6) | 2; 68 (68; 0) |
| F2 | 11/19 (57.9) | 17.4 (9.8-57.4; 13.6) | 55.2 (37.3-61; 10.5) | 19; 54 (37-61; 11) |
| F3 | 40/49 (81.6) | 15.5 (5.8-38.9; 8.9) | 45.3 (34-51.3; 6) | 32; 48 (47-51; 1) |
| F4 | 19/27 (70.4) | 12.4 (6.5-26.4; 6.0) | 33.6 (29.8-38; 4.1) | 21; 36 (30-56; 5) |
| F5 | 6/8 (75) | 7.6 (5.6-9.4; 1.6) | 13.5 (12.8-14.2; 1) | 9; 13 (13-14; 0.1) |

line 85

| | | | | |
|---|---|---|---|---|
| F1 | 0/7 | 0 | 55.7 (10-65.2; 20.2) | 5; 63 (62-65; 1.2) |
| F2 | 3/12 (25%) | 22.9 (14.1-34.7; 10.7) | 48.8 (47-50.4; 1.6) | 10; 48 (47-50; 1.5) |
| F3 | 0/8 | 0 | 26 (14.4-29.9; 7) | 6; 27 (14-30; 6.3) |

line 85 B16

| | | | | |
|---|---|---|---|---|
| F1 | 1/4 (25%) | 45.6 (45.6-45.6; 0) | 58.7 (58.4-58.9; 0.3) | 6; 59 (58-59; 0.2) |
| F2 | 2/5 (40%) | 26.7 (15.1-46; 16.8) | 49.4 (49.4-49.4; 0) | 2; 49 (49; 0) |
| F3 | 2/2 (100%) | 8.3 (6.8-9.8; 2.1) | | 4; 29 (29; 0) |

line 61

| | | | | |
|---|---|---|---|---|
| F1 | 0/5 | 0 | 59.7 (59.7-59.7; 0) | 1; 60 (60; 0) |
| F2 | 1/4 (25%) | 9.3 (9.3-9.3; 0) | 50 (49.5-50.3; 0.4) | 10; 50 (49-50; 0.4) |
| F3 | 0/4 | 0 | 28.5 (28.5-28.5; 0) | 4; 28 (28; 0) |

std. dev = standard deviation

The above demonstrates that the early genes of HPV are a causative agent for the development of skin tumors and establishes that the here-described animal model can serve as a tool for investigating anti-tumor agents as well as further causative agents for skin tumors.

In summary, transgenic mice were crossbred over five generations with FVB/N and B16 mouse strains. None of the HPV8-transgene negative mice developed lesions of the skin or any other organ. In contrast, more than 50% of HPV8-transgenic mice developed partially multifocal, benign tumors which were characterized by alopecia, hyperplasia, hyperkeratosis and ulcers.

The tendency for tumor development correlated with transgene loads. For three out of twenty (15%) post mortem examined mice histology revealed squamous cell carcinomas. This is the first experimental proof of the carcinogenic potential of an Ev-associated HPV-type in vivo. Via newly established real-time-RT-PCR protocols for HPV8-genes E2, E6 and E7 the expression of the integrate was confirmed. Highest expression levels were found for E2, followed by E7 and E6 with 10-30 fold higher levels in benign tumors compared to adjacent skin without pathological findings. In a mouse with a malignant tumor expression levels were comparable in the carcinoma and the skin without pathological findings, however, the levels were 10-50-fold higher than in normal skin of mice with benign tumors.

Whereas UV induced mutations in the tumor suppressor gene p53 are frequently detected in human skin carcinomas, mutations in p53 exons 4-9 via PCR and sequence analysis were observed neither in benign nor in malignant tumors. The notion, that the expression of viral genes in the mouse model is sufficient for the development of non-melanoma skin cancer emphasizes the high oncogenic potential of HPV8. The transgenic mouse-strains provide not only a valuable model for investigations of the functions of the early genes of HPV8 in vivo and for the development of antiviral strategies, it is also useful for the screening of potential anti-tumor drugs and allows the testing of tumorigenic potential.

1. A transgenic non-human animal comprising a recombinant nucleic acid molecule containing a nucleic acid sequence encoding at least one of the gene products of the early genes of one of the viruses of the Human Papilloma Virus Group B1, wherein said animal displays one or more clinical symptoms of a tumor.

2. The transgenic animal of claim 1, wherein said nucleic acid sequence is operably linked to a regulatory sequence directing its expression to epithelial cells.

3. The transgenic animal of claim 1, wherein said regulatory sequence comprises an epithelial cell specific promoter.

4. The transgenic animal of claim 1, wherein said regulatory sequence directs the expression of said nucleic acid sequence in basal epithelial cells.

5. The transgenic animal of claim 3 or 4, wherein said promoter is the keratin-14 promoter.

6. The transgenic animal of claim 1, wherein said at least one gene product comprises the E2, E6, or E7 genes of any one of the Human Papilloma Group B1 Viruses.

7. The transgenic animal of claim 1, wherein the Human Papilloma Group B1 Virus is Human Papilloma Virus type 8 (HPV-8).

8. The transgenic animal of claim 1, wherein said tumor is a tumor of epithelial cells.

9. The transgenic animal of claim 1, wherein said tumor is a skin tumor.

10. The transgenic animal of claim 1, wherein said animal is a rodent.

11. The transgenic animal of claim 10, wherein said rodent is a mouse or rat.

12. A method of producing a transgenic non-human animal displaying one or more clinical symptoms of a tumor, comprising
(a) introducing, into an embryo of a non-human animal, a recombinant nucleic acid molecule containing a nucleic acid sequence encoding at least one of the gene products of the early genes of a Human Papilloma Group B1 Virus;
(b) implanting the embryo into a female foster animal of the same species and allowing the embryo to develop normally until birth;
(c) screening the germline of the animal that develops from said embryo for presence of the nucleic acid construct; and
(d) mating the animal with at least a second animal produced by the method of steps (a)-(c) to produce offspring whose germline contains the nucleic acid construct.

13. A cell, or progeny therefrom, obtained from the transgenic animal of claim 1, wherein said cell, or progeny therefrom, is capable of expressing at least one gene product of the Human Papilloma Group B1 Virus early genes.

14. The cell of claim 13, wherein the cell is derived from a tumor of said transgenic animal.

15. The cell of claim 14, wherein said tumor is a skin tumor.

16. An animal model for tumor disorders comprising a non-human animal into which has been transplanted a tumor from a transgenic animal, wherein said tumor comprises cells having a nucleic acid molecule with a sequence that encodes at least one gene product of the early genes of a Human Papilloma Group B1 Virus stably integrated into the genome.

17. An animal model for tumor disorders comprising a non-human animal into which has been transplanted a tumor comprising one or more of the cells, or progeny therefrom, of claims 13, 14, or 15.

18. A method of screening a drug candidate for an effect on a tumor comprising:
(a) transplanting one or more tumor cells from a cell line of claims 13, 14, or 15 into an animal;
(b) administering said drug candidate to said animal; and
(c) evaluating the effect of said drug candidate on said transplanted tumor cells.

19. The method of claim 18, wherein said tumor is an epithelial tumor.

20. The method of claim 18, wherein said tumor is a skin tumor.

21. The method of claim 18, wherein said drug candidate is evaluated for an ability to inhibit the growth of a tumor developed from said one or more transplanted cells.

22. The method of claim 18 wherein said drug candidate is evaluated for an ability to inhibit the formation of a tumor developed from said one or more transplanted cells.

23. A method of screening a drug candidate for an effect on a tumor comprising
(a) administering said drug candidate to the transgenic animal of claim 1; and
(b) evaluating the effect of said drug candidate on a tumor present in said transgenic animal.

24. The method of claim 23, wherein said tumor is an epithelial tumor.

25. The method of claim 23, wherein said tumor is a skin tumor.

26. The method of claim 23, wherein said drug candidate is evaluated for an ability to inhibit the formation said tumor.

27. The method of claim 23, wherein said drug candidate is evaluated for an ability to inhibit the growth said tumor.

28. A method of screening a drug candidate for the treatment of a tumor comprising
(a) contacting one or more cells of claims 13, 14, or 15 with said drug candidate in vitro; and
(b) evaluating the effect of said drug candidate on said one or more cells.

29. The method of claim 28, wherein said drug candidate is evaluated for an ability to inhibit the growth of a tumor derived from said one or more cells following transplantation of said one or more cells into an animal.

30. The method of claim 28, wherein said drug candidate is evaluated for an ability to inhibit the growth of said one or more cells.

31. The method of any one of claims 18, 23, or 28, wherein said drug candidate is provided as a collection of compounds.

32. The method of claim 31, wherein the number and/or diversity of compounds within said collection is successively reduced in repeated screening rounds.

33. An anti-tumor molecule identifiable by any one of the methods of claims 18 to 32.

34. The method of any one of claims 18, 21, 28, or 30, wherein said drug candidate is an antibody.

35. The method of claim 34, wherein said antibody is an antibody conjugate.

36. The method of any one of claims 18, 21, 28, or 30, wherein said drug candidate is a small molecule.

37. The method of any one of claims 18, 21, 28, or 30 further comprising the step of identifying a candidate that has the ability to induce death or apoptosis of cancer cells.

38. A recombinant nucleic acid molecule comprising a nucleic acid sequence encoding at least one gene product of the early genes of a Human Papilloma Group B1 Virus under the control of a regulatory sequence directing its expression in epithelial cells.

39. The recombinant nucleic acid molecule of claim 38, wherein the regulatory sequence comprises a keratin-14 promoter.

40. The recombinant nucleic acid molecule of claim 39, wherein said promoter is a human promoter.

41. An oligonucleotide primer comprising the sequence of any one of SEQ ID NOS: 7 to 12.

42. A method of screening agents for a cancer promoting or protecting effect comprising
(a) contacting one or more cells of claim 13, 14, or 15 with a candidate agent; or
(b) contacting an animal of claim 16 or 17 with a candidate agent; and
(c) evaluating the effect of said agent on said cells or the animal.


SEQUENCE LISTING (12 sequences)

SEQ ID NO: 1 - DNA, 5116 bp, Human papillomavirus type 8
gatccaacggtaagtttcatcagtgtaccaggtgcggtatgaaaatttcttaatcataagttgttattgccaacaaccatcgtctatagcatgtttttgcctgtatcgttttcgatcacaccatattgtatattaaataaataaataaatatatatatatattgttacaatgctgtgacttgtgcaattttcctaagcaaatggacgggcaggacaaggcttcatatttagacactaataaggacgagctaccctctactattaaagagttagctgcggctttaggtattccattgcaggactgttcagtaccgtgcaacttttgtggtaactttttggatttcttagaactgtgtgagtttgacaaaaagagactgtgcctaatttggaaaaattacgttgttactgcgtgttgtcgttgttgttgtgtagcaaccgcaacgtttgaatttaatgaatattatgagcaaactgtgctaggcagagatattgaattagctacaggacgttcaatttttgagatagacgttaggtgtcaaaactgcttgtcatttttggatatcatagagaaattagattgctgtgggagaggccgtccctttcataaagttagaggaggctggaaaggagtttgcaggctttgtaagcatttgtatcatgattggtaaagaggtcactgtgcaagattttgtgttgaagttaagtgagatacaacctgaagtgttaccagttgacctgctttgtgaagaggaattaccaaacgaacaggaaacggaggaggagctagacatcgaaagaactgtattcaaaattgttgcaccgtgtggctgcagctgctgtcaggtcaagctacgtctttttgtcaacgcaactgattcgggtatcaggacctttcaagaattgctgttcagagacctacagcttctgtgtcctgagtgccgcggtaactgcaaacatggcggatcataaaggtagtacatctaaagaagggttaagtgagtggtgtattttggaagctgaatgtagtgatgtagacaatgattttgaacaattatttgagcgagatacagactcagatatttcggacttattagataattgtgacctggatcagggaaattctctggaactatttcatcaacaggagtgtgagcagagcgaggagcaattacaaaaactaaaacgaaagtatcttagtcctaaagctgtcgcgcagctcagtccgcggctccagtcaatatcactgtcacctcagcagaaatccaagcgaaggctctttgcagagcaggacagcggagtcgagctaactcttaacaatgaagctgaagatgtttctcatgaggtggaggtaccggctatagactctcggccggaagatgagggaggatcaggggctttagatattgactatacagcattgttgcggtccagcaacacaaaggccacattaatggcaaaatttaaagaggcatttggggatggctttaatgaactaacacgccaatttaaaagttacaaaacttgctgtaactattgggttgtagctgcttatgcagtgcatgatgtatatgaaagctcaaagcagttattgcaacagcattgtgattatatttgggttagaagtatagctgcaattacattatatctattgtcttttaaggcgggaaaaaatcgcggtactgtgcataaactaatgacctcaatgctaaatgtgcaagagcagcagatattgtctgagcctcctaaattgagacacactgctgctgctttgttttggtataaaggaggaatggggacaggaacattcacgtatggttcataccctgattggattgcacatcaaacaattcttggccatcaaagtgctgaagcaagcacctttgatttttctgtaatggtacaatgggcatttgataataatcattttgaggaggccgacattgcttatggatatgcaaaactagccccagaagatgctaatgctgtagcctggcttgctcataatagccaagctaaatttgtaagagagtgtgcagcaatggttcgattttacaaaagggggcaaatgagagaaatgaccatgtctgagtggatatatacaaggatcaatgaggttgaaggagaggggcattggtcttccatagtaaaatttgtaaggtaccagggtataaattttattacatttttagctgccttaaaagattttttgcattccgtacctaagcgaaactgtttgttaatctatggtccacctaatactgggaaatctacatttgctatgtcattaatacaggtgctgaagggtagagtattatcatatgtaaattccaaaagccaattttggttacaacctcttggtgactgtaaaatagcactgttagatgatgtgacagacccctgctggctgtacatggatacatttctgcgaaatggattagatgggcatgttgtgtcattagattgtaaatataaagcacccatgcaaattaaatttcctccccttttgttaacttccaatattaacctgcatgaggaggctaactatagatatttacatagcagagtcagaggatttgaatttccaaatccttttccaatgaaaccagacaatacacctgaatttgaattaactgaccaaagctggaaatctttttttgcaaggctttggacacaattagagctgagtgatcaagaagacgagggcgaacatggagaatctcagcgagcgtttcaatgttctgcaagatcagctaatgaacatttatgaagctgcagaacaaacacttgaggcacagattgcgcattggctgcttttgcgaaaagaagctgtgttgctatatttcgctaggcaaaaaggcatcaccaggattggatatcaacctgtgccgccactagcagtgtctgaagcaaaagccaagcaggctataggaataatgttgcagctgcagtcactacagaaatctgagtttgcagatgagccttggacattagtggacacaagtatagagacttataagaatgctccagaaaatcattttaaaaaaggcgccacacctgtggaggtgatatatgataaacagcctgacaatgccaatgtatacactatgtggaagcacatttactacactgatgcagacgataagtggcacaaaaccaccagtggggttaaccagactggcatatactatatgcaaggcagcttcagacattattatgttgtgtttgctgatgatgcacgtagatatagtgctactggagaatgggaagtaaaaattaataaggacactgtgtttgctcctgttaccagctccaccccccccggatcaccaccaggacaagcagacacagacaccgccgccaagacccccaccacctccgctgactccacgtccagacagcagcggtcccctgcaaaacagccacaacaaaccgaaaccaaaggacgaaggtacgggagacggccgtccagcagaacaagaccgcaaaaagagcagaggcgatcaaggtcgcgacaccgcaccaggtctcgctcccggtcgctctcccgggttagggccgttggctccaccaccgtatccaggtccaggtcctcgtcgctcaccaaggcagttcggccccggtccagatcgcgatccagaggacgggctacagccacctctaggcgaagggcaggtcgagggtcacccaggcgacggcgatcaacctcaaggtcaccctccaccaacaccttcaaacggtcacaaaggggaggagggagacggggaagaggaaggggcagtagggggagacgggaacgatcatcctccacctcccccacccccaccaaacgttcaagaggggagtcttctaggttgcgtggggtctctccttctgaagtgggaagatcagttcaatctgttagtgcaaaacatacagggcgacttggaagactactggacgaagctatcgaccccccagtaatattggttcgaggggaggcaaacacattaaaatgctttcgcaacagagctagagttagatatagaggactgttcaaatactttagcaccacgtggtcatgggtggccggcgatagcactgagcgtctaggcaggtccagaatgctcattctgtttacttcagctggccaaagaaaggactttgatgagactgtgaaatacccgaagggagttgatacatcttatggtaacctggacagtctataacattactaacgctgccttgctactaacacactaacatattcccatttgctttttactatattttttaattgtatactgctatggcgcgtgctagacgggtcaagagagactctgtcacacacatttatcaaacctgcaaacaggcaggtacatgcccccctgatgttattaataaagttgagcaaacaacagttgctgacaatattttaaaatatggcagtgctggtgtattttttggaggccttggtataggtaccggccgtggtacagggggtgttactgggtacacgccattaagtgaggggcctggtatccgtgttggaaatactcccacagtggtacggccctcacttgttccagaagcggtaggtcctatggacatactgccaatcgacactattgaccctgtagagccttcagtctcctctgtggtgccactcactgaatcttcaggagccgacctacttccaggggaagttgagacaattgctgaaattcaccctgtacctgaaggccccaccattgattcacctgtggtgacgacaagcaaagggtcaagtgccattttagaggtggctcctgagccaacccctcctacccgtgtcagagtttcacgtacacaatatcacaacccttcttttcaaatcataactgattccacacctacacaaggagaaagttctctagcagaccacattttagtgacttctggctctggaggccagactataggtagtgacataacagatgtcatagaactgcaagaatttcctagtaggtattcatttgagattgatgaacctacaccaccacggcaaagtagcacacctattgagagacctcaagtagttggtagacgtagaggtatttctttaacaaatagacgactgatacagcaggtggctgttgag

SEQ ID NO: 2 - DNA, 2008 bp, Homo sapiens
cgggctccggagcttctattcctgggccctgcataagaaggagacatggtggtggtggtggtgggtgggggtggtggggcacagaggaagccgatgctgggctctgcaccccattcccgctcccagatccctctggatatagcaccccctccagtgagcacagcctccccttgccccacagccaacagcaacatgcctcccaacaaagcatctgtccctcagccaaaacccctgttgcctctctctggggaaattgtagggctgggccagggtggggggaccattctctgcagggagattaggagtgtctgtcaggggcgggtggagcggggtggggccctggcttactcacatccttgagagtcctttgctggcagatttggggagcccacagctcagatgtctgtctcagcattgtcttccaagctcctaggccacagtagtggggcgctcccttctctggcttcttctttggtgacagtcaaggtggggttgggggtgacgaagggtcctgcttctcttctaggagcagttgatcccaggaagagcattggagcctccagcaggggctgttggggcctgtctgaggagataggatgcgtcaggcagccccagacacgatcacattcctctcaacatgcctgccggggtctgtggagccgaggggctgatgggagggtggggtgggggccggaagggtttgctttgggaggttgtctgggagattgctgaagttttgatatacacacctccaaagcaggaccaagtggactcctagaaatgtcccctgacccttggggcttcaggagtcagggaccctcgtgtccacctcagccttgcccttgcacagcccagctccactccagcctctactcctccccagaacatctcctgggccagttccacaaggggctcaaacgagggcacctgagctgcccacactagggatgttctgggggtctgagaagatatctggggctggaagaataaaaggcccccctaggcctgttcctggatgcagctccagccactttggggctaagcctgggcaataacaatgccaacgaggcttcttgccatactcggtttacaaaaccctttacatacattgtcgcattggattctcagagctgactgcactaagcagaatagatggtatgactcccactttgcagatgagaacactgaggctcagagaagtgcgaagccctgggtcacagaggcgtaaatgcagagccaggacccacctgaagacccacctgactccaggatgtttcctgcctccatgaggccacctgccctatggtgtggtggatgtgagatcctcaccatagggaggagattagggtctgtgctcagggctggggagaggtgcctggatttctctttgatggggatgttggggtgggaatcacgatacacctgatcagctgggtgtatttcagggatggggcagacttctcagcacagcacggcaggtcaggcctgggagggccccccagacctccttgtctctaatagagggtcatggtgagggaggcctgtctgtgcccaaggtgaccttgccatgccggtgctttccagccgggtatccatcccctgcagcagcaggcttcctctacgtggatgttaaaggcccattcagttcatggagagctagcaggaaactaggtttaaggtgcagaggccctgctctctgtcaccctggctaagcccagtgcgtgggttcctgagggctgggactcccagggtccgatgggaaagtgtagcctgcaggcccacacctccccctgtgaatcacgcctggcgggacaagaaagcccaaaacactccaaacaatgagtttccagtaaaatatgacagacatgatgaggcggatgagaggagggacctgcctgggagttggcgctagcctgtgggtgatgaaagccaaggggaatggaaagtgccagacccgccccctacccatgagtataaagcactcgcatccctttgcaatttacccgag

SEQ ID NO: 3 - DNA, 640 bp, Oryctolagus cuniculus
tgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattc

SEQ ID NO: 4 - DNA, 486 bp, Homo sapiens
cctaggaggccccccgtgtggacacagatcccactggaagatcccctctcctgcccaagcacttcacagctggaccctgcttcaccctcaccccctcctggcaatcaatacagcttcattatctgagttgcataattctcgcctctctctggtcattgttaggagtgggggtggggagaaagtgggagagcatctctttggagcttgtcatgcacctggctatggcccctgggactgggagaaaagtcctgggggtgggttgggctcaggtcccaggatatctttcgccatctcagaagacacagatagatgtgtgtaccaggtcatatgtggtgtctcctagggtacggagggatattcattcatttactcactcattttcatgtgtgtccattcattcaccagatattgagtgcctctatgtcaggcactatgttaggttaaggattcctgatgtttttgtgtatcagggattccttggagaat

SEQ ID NO: 5 - DNA, 22 bp, Artificial Sequence
  synthetic
caattttcctaagcaaatggac

SEQ ID NO: 6 - DNA, 25 bp, Artificial Sequence
  synthetic
cactacattcagcttccaaaataca

SEQ ID NO: 7 - DNA, 22 bp, Artificial Sequence
  synthetic
gcggctttaggtattccattgc

SEQ ID NO: 8 - DNA, 25 bp, Artificial Sequence
  synthetic
gctacacaacaacaacgacaacacg

SEQ ID NO: 9 - DNA, 25 bp, Artificial Sequence
  synthetic
cctgaagtgttaccagttgacctgc

SEQ ID NO: 10 - DNA, 22 bp, Artificial Sequence
  synthetic
cagttgcgttgacaaaaagacg

SEQ ID NO: 11 - DNA, 18 bp, Artificial Sequence
  synthetic
aacagccacaacaaaccg

SEQ ID NO: 12 - DNA, 20 bp, Artificial Sequence
  synthetic
cgtatccaggtccaggtcct